(19) United States
(12) Patent Application Publication
(10) Pub. No.: US 2012/0149689 A1
(43) Pub. Date: Jun. 14, 2012

(54) METHOD OF WEANING FROM HORMONAL TREATMENT OF HORMONE DEPLETION INDUCED VASOMOTOR SYMPTOMS

(76) Inventors: Edward Baker, Rahway, NJ (US); Simon Johannes van den Berg, Rahway, NJ (US)

(21) Appl. No.: 13/403,508

(22) Filed: Feb. 23, 2012

Related U.S. Application Data
(63) Continuation of application No. 11/714,301, filed on Mar. 6, 2007, now abandoned.
(60) Provisional application No. 60/779,599, filed on Mar. 6, 2006.

(51) Int. Cl. A61K 31/55; A61P 43/00

(52) U.S. Cl. 514/214.02

(57) ABSTRACT

The invention provides a method to effectively wean a woman from hormonal therapy for treatment of vasomotor symptoms by reducing the dosage of the hormonal agent to zero, while a non-hormonal drug in an amount, which is therapeutically effective for treating vasomotor symptoms, is administered or initiated and continued for one or more periods of durations determined by assessment of the effect of a treatment free period, which follows each period of continuous non-hormonal drug treatment.

The invention pertains to an improved method of weaning from hormonal treatment of hormone depletion induced vasomotor symptoms by using administration of non-hormonal drug therapy.

At a recent NIH sponsored meeting which reviewed the “state of the science for the management of menopause related symptoms” in March 2005 it was concluded that: “Based on review of currently available cohort and cross-sectional population studies, vasomotor symptoms and vaginal dryness are symptoms most consistently associated with the menopausal transition. Sleep disturbance, somatic complaints, urinary complaints, sexual dysfunction, mood, and quality of life are inconsistently associated.” and: “Estrogen, in either opposed or unopposed regimens, is the most consistently effective therapy for vasomotor symptoms, and demonstrates benefit in most trials evaluating urogenital symptoms. Some, but not all trials evaluating sleep, mood and depression, sexual function, and quality of life outcomes also report benefit with estrogen compared to placebo.” and: “For women with breast cancer, results of 15 randomized controlled trials indicate that clonidine, venlafaxine, and megestrol acetate are associated with significantly improved measures of hot flashes, and vitamin E, black cohosh, isoflavones, magnets, and fluoxetine are not. Results for nonvasomotor outcomes are mixed.” and: “In order to fill evidence gaps, future research could focus on: [ - - ] Trials demonstrating how to discontinue estrogen when symptoms subside, including the effectiveness of tapering doses and/or replacing with other therapies including non-drug interventions.”

Clearly there is much to be discovered about how vasomotor symptoms work and what to optimally do about them. Currently, the gold standard for reduction of vasomotor symptoms associated with menopause is estrogen therapy. European and American regulatory authorities, and groups such as the American College of Obstetrics and Gynecology and the North American Menopause Society all currently recommend using the lowest appropriate estrogen dose and limiting the length of therapy to that which is necessary to meet treatment goals. What is lacking is advice on how to wean a patient from estrogen therapy. It could be that estrogen therapy simply delays the hot flashes a patient experiences at the time of menopause to a time when hormonal therapy is discontinued. This is supported by the experience of many following the sudden self-imposed withdrawal of women from hormonal therapy in light of the recent reports regarding outcomes from the Women's Health Initiative in 2002. Many of these women experienced significant vasomotor symptoms to the extent that they eventually restarted estrogen therapy. Many women will be able to discontinue hormones on their own, while others will have substantial symptoms from estrogen withdrawal and will either be successful quitters, but suffer or will return to therapy. (See D. Grady, Obstet Gynecol 2003:102(6);1233-1239). The latter are the two populations that will benefit from our invention.

It is a long-standing desire to be able to help patients quit hormones. The use of paroxetine and various other selective serotonin re-uptake inhibitors as an alternative to hormonal therapy are well known to the average gynecologist in the US. Yet despite this, it had not been thought of to use these drugs to wean a patient from hormonal therapy. In the NIH meeting mentioned above, options of either weaning a patient from estrogen, over some course of time, or weaning a patient to another medication, which she would be able to take with less risk, was considered, but there was no thought of using an agent to assist in moving a patient from estrogen to no therapy whatsoever.

The present invention provides a method to effectively wean a woman from hormonal therapy for treatment of vasomotor symptoms by reducing the dosage of the hormonal agent to zero, while a non-hormonal drug in an amount, which is therapeutically effective for treating vasomotor symptoms, is administered or initiated and continued for one or more periods of durations determined by assessment of the effect of a treatment free period, which follows each period of continuous non-hormonal drug treatment. The problem with hormonal drug therapies is that stopping the administration does not have an immediate consequence of depletion of the hormonal influence, in view of the protracted action of hormonal therapy. The slow offset of action may have to do with the slow fading of the physiological consequences of hormonal therapy, possibly related to the changes needed in genetic expression of genes in the cells of the treated person. Hormonal therapies act via nuclear receptors which are believed to effectuate their actions slowly by modulating the genetic expression in an organism. In contrast, non-hormonal drug therapies which are not based on interacting with nuclear receptors, but on influencing receptors on cell membranes, for example G-protein coupled receptors, or other rapidly responding receptors, are having rapidly reversible effects, which fade at about the same rate by which the drug is cleared from the body. This enables an efficient test for assessment of the need for continued treatment of the vasomotor symptoms, without heavy burden for the person undergoing treatment. The drug treatment periods may last 1 to 6 months and the drug free periods 1-7 days. The duration of the treatment period is related to the shortest time a person under treatment adapts to the new state of non-hormonal treatment. The phasing out of hormonal treatment and the replacing treatment of the vasomotor symptoms with non-hormonal treatment is supposed to effectively amount to treatment of hormonal depletion symptoms, which may turn out to have been completed in the short mentioned period of 1-6 months. If the person turns out not to have been adapted to the hormone depleted state, it will be rapidly evident by full re-occurrence of the symptoms during the treatment free days. Treatment can be re-instated for a subsequent period of treatment and the burden to the treated person will not be too heavy. After each treatment free period a decision can be made to completely stop treatment or to lower the dose of the treatment. The alternating periods of treatment and non-treatment may vary in their duration depending on the circumstances of the treated person and results of earlier assessments of the consequence of 1-7 days of non-treatment.

An embodiment of the invention is a treatment with non-hormonal drug treatment for 2-4 months followed by a short period of non-treatment of 1-4 days. Other embodiments of the invention are other sequences of periods of 1-6 months alternating with periods of 2-4 days non-treatment. Other variants are that each three months of treatment is followed by 2, 3, 4, 5 or 6 days of non-treatment. Each of the 2, 3, 4, 5 or 6 days non-treatment periods can be combined with a preceding treatment period of 1, 2, 3, 4, or 5 months and so each combination or variable sequence may arise in using the invention.

Basic to the invention, but without binding to such a theory, is that a period of a few months is needed and sufficient to adjust to lower levels of hormonal action, leading to gradual decline in the frequency and severity of vasomotor symptoms.

The success rate of the weaning method can be further improved by selecting postmenopausal women. This characteristic can be established according to the usual manners available to the clinician, such as the time period lapsed since the last menstruation, the age of the woman, in particular an age of 55 and older commonly indicates postmenopausal state, etc. The determination can be verified after weaning by measuring endogenous FSH (follicle stimulating hormone), which will no longer be fluctuating, but rather be a constant high plasma level (>40 mIU/mL).

The terms in this description are used with the following meaning:

“non-hormonal” is a mechanism of action of a drug therapy of vasomotor symptoms not based on activation of the estrogen receptor.
“weaning” is the termination of hormonal therapy.
“weaning agent” is a non-hormonal drug given to assist weaning
“hormonal therapy” is a treatment against undesirable effects caused by decline in endogenous estrogens in a woman based on reinstatement of activation of estrogen receptors, for example by estrogen therapy or hormone replacement therapy or prescription of regimes to maintain the monthly cycle in a woman.
“non-hormonal drug” is a drug not having a hormonal mechanism of therapeutic action.
“non-hormonal drug therapy” is a therapy for countering one or more undesirable effects caused by decline in endogenous estrogens with a non-hormonal agent.
“A therapeutically effective amount of a non-hormonal drug” is an amount of the non-hormonal drug which prevents to a large extent one or more of the undesirable effects caused by decline in endogenous estrogens in a woman.
“Hot flash” is a sensation of heat or burning which usually starts in the upper torso and head. It is probably the most distressing symptom of menopause and is experienced by approximately 80% of menopausal women.
“Menopause”—the final menstrual period, usually diagnosed retrospectively after at least one year without menstruation. It is, though, commonly understood, and used here in that sense, that the expression ‘menopausal women’ refers to women who are in a period of their life that is transitional between mature female physiological functioning and postmenopausal functioning. In that sense the term ‘vasomotor symptoms (hot flashes) associated with menopause can be understood.

It is an essential means of the invention that the dosage of the hormonal agent is reduced while giving or initiating non-hormonal therapy for vasomotor symptoms. The reduction in dosing of the hormonal treatment can be immediate by termination of any administration of the hormonal agent or gradual over a period of a number of months or weeks, for example two weeks, during which the dosage is reduced stepwise. If administration of non-hormonal therapy has not already been started shortly, that is a few days, before reduction of the hormonal therapy, the non-hormonal therapy should start soon, say, within days or weeks after having reduced the administration of a hormonal agent.

As compound for non-hormonal treatment any compound effective against hot flash can be selected, such as mirtazapine or its enantiomer (S)-mirtazapine. Alternatively, venlafaxine or one or a mixture of its enantiomers or the desmethyl analog of all these can be selected. For the treatment of menopausal symptoms mirtazapine is to be administered to a women in a suitable daily dose, which will be in the range of from 0.5 to 140 mg, calculated on the weight content of base, per recipient per day, preferably in the range of 1 to 20 mg and most preferably in the lower range of 1-10 mg or even below 5 mg, such as 3 or 4.5 mg of the base per recipient per day. In general, parenteral administration requires lower dosages than other methods of administration which are more dependent upon absorption. However, the daily dosages are between 0.01 and 1.5 mg/kg body weight of the recipient and the oral route of administration is preferred.

In the case of tolerance development, treatments can be further optimized by increasing the dose up to 5 times in the course of a chronic treatment in humans. The desired dose may be presented as one, two, three or more sub-doses administered at appropriate intervals throughout the day.

Mirtazapine is known to existing in two enantiomers in S- or R- configuration. The drug can be used for the purpose of the invention as racemic mixture or as one enantiomer substantially free of the other enantiomer. The S-mirtazapine is preferred as active ingredient for the method according to the invention. The compound can be used for the purpose according to the invention as a free base or as one or more of the commonly accepted acid addition salts. Such compounds can be used in pure form or in admixture with pharmaceutical excipients.

The amount of mirtazapine, S-mirtazapine or R-mirtazapine, also referred to herein as the active ingredient, which is required to achieve a therapeutic effect will, of course, vary with the particular compound, the route of administration and the age and other conditions of the recipient. The amounts of mirtazapine defined in this description refer to the amount of the base of mirtazapine.

While it is possible for the active ingredient to be administered alone, it is preferable to present it as a pharmaceutical formulation. Accordingly, the present invention further provides a pharmaceutical formulation for use in the treatment according to the invention, together with a pharmaceutically acceptable carrier thereof and optionally other therapeutic agents. The carrier must be “acceptable” in the sense of being compatible with the other ingredients of the formulation and not deleterious to the recipients thereof. The invention further includes a pharmaceutical formulation, as hereinbefore described, in combination with packaging material suitable for the pharmaceutical formulation, said packaging material including instructions for the use of the pharmaceutical formulation in the treatment of hot flush.

Formulations include those suitable for oral or vaginal administration. The formulations may be prepared by any methods well known in the art of pharmacy. Such methods include the step of bringing into association the active ingredient with the carrier which constitutes one or more accessory ingredients. Such accessory ingredients include those conventional in the art, such as, fillers, binders, diluents, disintegrants, lubricants, colorants, flavoring agents and wetting agents.

Formulations suitable for oral administration may be presented as discrete units such as tablets or capsules each containing a predetermined amount of active ingredient as a powder or granulate, as a solution or suspension.

Formulations, which are parenteral (for example subcutaneous) may also be presented in a suitable sustained release form for drug delivery over a number of days or weeks.

Mirtazapine, S-mirtazapine and R-mirtazapine can be prepared in several manners, Mirtazapine may be prepared using the method described in U.S. Pat. No. 4,062,848, possibly followed by purification to an enantiomerically pure form. Enantiomerically pure mirtazapine can also be obtained by stereoselective synthesis (WO 2005/005410).

### EXAMPLE

The following is a randomized, double-blind placebo-controlled parallel example of weaning from long-term (>2 years) hormonal therapy during a period of 12 weeks of S-mirtazapine maleate treatment in menopausal women, after which period treatment is stopped in order to assess tolerance of the treated person to non-treatment.

### Blinded and Randomized Medications:

S-mirtazapine maleate in daily doses of 2 tablets. One tablet of S-mirtazapine maleate consists of 4.5 mg active entity (active entity is the base part of the salt of (S) enantiomer of mirtazapine).

Placebo tablets in daily doses of 2 tablets.

Subjects are instructed to take two tablets of the medication orally per day, in the evening prior to sleep, starting at the first dosing day (=Day 1). Subjects receive once daily evening doses of 9 mg S-mirtazapine maleate or placebo during 12 weeks.

### Observation Parameters:

The difference in average daily composite score of hot flushes between the S-mirtazapine maleate and placebo group from first until last treatment with medication.

The difference in average daily severity score and frequency score of hot flushes between the S-mirtazapine maleate and placebo group from first until last treatment with test medication.

The difference in average daily composite score, severity score and frequency score of hot flushes between the S-mirtazapine maleate and placebo group from the start of treatment until last follow-up.

The difference in average daily composite score, severity score and frequency score of hot flushes between the S-mirtazapine maleate and placebo group from last treatment with medication until last follow-up.

The difference in percentage of and time to return to hormonal therapy between the S-mirtazapine maleate and placebo group from the start of treatment until last follow-up.

The safety and tolerability of once daily treatment with S-mirtazapine maleate.

### Design:

A single centre, randomized, double-blind, placebo-controlled parallel two-group multiple dose study in 20 menopausal women. Subjects fulfill all the inclusion criteria and none of the exclusion criteria.

Test medication is administered once daily in the evening on Days 1-84.

### Methods of Evaluation

### Screening

After informed consent being given in writing, screening of eligibility is performed in accordance with an inclusion/exclusion list. Subjects are asked for their medical history, including gynaecological history, and have a physical examination including breast examination, vital signs, routine haematological, biochemical and urinary investigations as well as a gynaecological examination including a cervical smear if a report of a normal smear no older than 9 months is not available at screening. Subjects are screened for selection within 28 days prior to the start of treatment with the test medication. Eligible subjects fulfill all the inclusion criteria and none of the exclusion criteria.

### Ambulant Visits to the Treatment Centre

All subjects visit the treatment centre for screening, at Baseline, approximately 4, 8 and 12 weeks after first dosing and for three follow-up visits: 30-35 days, 60-65 days and 90-95 days after the last medication intake.

### Efficacy

Frequency of mild, moderate and severe hot flushes and severity of hot flushes as documented daily in paper diaries from screening until last follow-up. Return to hormonal therapy from first treatment until last follow-up, as documented by questioning by the investigator at each visit.

### Safety

Observations for clinical safety include routine laboratory assessments, vital signs (heart rate, blood pressure), body weight, and documentation of any (serious) adverse event reported either spontaneously or after questioning during the whole test period. Observations for laboratory safety include routine haematology and biochemistry.

### Analysis of Test Results

The average of the logarithm of the area under the curves (AUC) of the daily composite hot flush scores (divided by the actual time span), frequency and severity scores are analyzed. The incidence of return to hormonal therapy is calculated. Time to return to hormonal therapy, demographics and safety data are analysed.

Twenty subjects are included in the test: 10 subjects receive S-mirtazapine maleate treatment and 10 subjects receive placebo treatment.

### Inclusion and Exclusion Criteria

### Inclusion Criteria

1. Women who have maximally 2 moderate to severe hot flushes per day during the screening period and who are currently using hormonal therapy (for the treatment of hot flushes) for a duration of >2 years and who are willing to stop their treatment;

2. Age ≧45 and ≦60 years;

3. The initial reason to start hormonal therapy was the occurrence of intolerable hot flushes;

4. At least one attempt was undertaken to stop hormonal therapy, which was unsuccessful due to the recurrence of intolerable hot flushes;

5. Body mass index (BMI) ≧18.0 and ≦32.0 kg/m²;

6. Able and willing to sign the Informed Consent Form prior to screening evaluations;

7. (History of) good physical and mental health as determined by history taking, breast, gynecological, physical and laboratory examinations, and vital signs recordings.

### Exclusion Criteria

1. History of sensitivity/idiosyncrasy to S-mirtazapine maleate, mirtazapine or chemically related compounds or excipients which may be employed in the study or to any other unknown drug used in the past;

2. Lack of compliance (including stop attempt(s)) with hormonal therapy of more than 30 days during the past 2 years;

3. Lack of compliance (including stop attempt(s)) with hormonal therapy of more than 4 days during the last 6 months;

4. History or presence of any malignancy;

5. Any clinically unstable or uncontrolled disease that would put the subject at safety risk or mask measure of efficacy;

6. History of seizures or epilepsy;

7. History or presence of clinically significant depression or other psychiatric disorder which, in the opinion of the investigator, might compromise or confound the subject's participation in the test;

8. Abnormal, clinically relevant vaginal bleeding;

9. Abnormal cervical smear test results (corresponding to Pap III and higher, including LSIL, HSIL, CIN1 and higher). A cervical smear must have been performed within the last 9 months prior to screening, otherwise it must be done before inclusion into the test;

10. Abnormal laboratory, vital signs or physical findings at screening (and at baseline) considered clinically relevant by the investigator;

11. Subject is not able to accurately record drug accountability and/or hot flushes information according to the opinion of the investigator;

12. High blood pressure (sitting systolic BP >170 mmHg and/or diastolic BP >100 mmHg);

13. Any of the following treatments within the last 4 weeks prior to screening (and up to and including first dosing):
any drugs that effect 5-hydroxytryptamine (e.g. tricyclic antidepressants, SNRIs, SSRIs, MAO-inhibitors, mirtazapine)
antianxiety drugs, antipsychotics
coumarin-derivatives
α-adrenergic agents (e.g. clonidine, methyldopa)
β-blockers (e.g., propranolol)
dopamine agonists/antagonists (e.g., veralipride, bromocriptine, domperidone)
opiates, barbiturates
raloxifene
gabapentin
any preparation intended to treat climacteric or CNS symptoms (e.g. Black Cohosh, St. John's Wort, isoflavone supplements)
hepatic microsomal enzyme-inducing drugs or drugs known to affect or interfere with the pharmacokinetics of mirtazapine;

1. Any condition or disease that could affect or interfere with the pharmacokinetics of mirtazapine;

2. History of or current abuse of drugs or solvents and/or history of or current abuse of alcohol (i.e. daily use of more than 2 units of alcohol per day);

3. Inability to understand the nature and extent of the test and the procedures required;

4. Participation in an investigational drug study within 90 days prior to the first dose.

Following assessment of the effect of stopping treatment after three months the option to reinstate treatment with S-mirtazapine maleate is offered to women who still find the symptoms unacceptable. This starts a second cycle of three months of S-mirtazapine treatment, after which again treatment is stopped for at least two days.

### More Detailed Description of the Method of This Example:

### Screening

Subjects are informed, orally and in writing, about the purpose, procedures, and general risks of participating in this test. After signing the informed consent form, the subject is evaluated for eligibility to participate in the test.
The following screening activities can be performed:

Demographic data and other subject characteristics

General medical and gynaecological history

General physical, gynaecological and breast examination including cervical smear (if applicable)

Recording of vital signs

Blood and urine sampling for routine laboratory assessments

Recording of current used oral contraceptives, hormonal therapy

Recording of pre-test medication

Recording of adverse events

After having satisfied the inclusion and exclusion criteria the subject is trained in completion of paper diaries. The subject is instructed to record the severity and frequency of hot flushes for a period of at least seven complete days and to continue to make diary entries until she returns for the Baseline Visit, even if the time interval between Screening and Baseline exceeds eight days. Subjects are trained to record the number and severity of hot flushes occurring during the day prior to going to sleep and to record the number and severity of hot flushes occurring during the night in the morning at waking up.

Note: in order to get the most objective data, the subjects are not informed on the maximum number of hot flushes needed to participate in the test.

### Baseline Visit

Diary entries are reviewed for accuracy. Subjects who experience no more then the maximum number of hot flushes allowed during at least 7 complete days immediately preceding the Baseline Visit and meet all other inclusion/exclusion criteria, are treated with test medication.

After completion of all baseline assessments, all inclusion and exclusion criteria need to be checked. A medication number is assigned according to Menox procedures to each subject fulfilling all entry criteria. Subjects are instructed to stop their hormonal therapy and start test medication on the day of the Baseline Visit (Day 1), in the evening prior to sleep.

The following assessments are performed during the Baseline Visit:

Inclusion and exclusion checklist

Collect diary card baseline period

Recording of vital signs

Recording of pre-test medication

Recording of (serious) adverse events

Dispense of test medication (Bottle no. 1) and diary.

### Treatment Period

Week 4 Visit after a treatment period of four weeks (between Day 24 and Day 34), the following assessments are performed:
Check on completion of daily diary entries
Collect completed diary card and left over medication
Recording of vital signs
Blood laboratory assessment
Recording of concomitant medication
Recording of any (serious) adverse events
Check on relapse to hormonal therapy
Drug accountability and check for compliance
Dispense of test medication (Bottle no. 2) and diary.

Week 8 Visit after a treatment period of eight weeks (between Day 52 and Day 62), the following assessments are performed:
Check on completion of daily diary entries
Collect completed diary card and left over medication
Recording of vital signs
Recording of concomitant medication
Recording of any (serious) adverse events
Check on relapse to hormonal therapy
Drug accountability and check for compliance
Dispense of test medication (Bottle no. 3) and diary.

Week 12 Visit after a treatment period of four weeks (between Day 85 and Day 91), the following assessments are performed:
Check on completion of daily diary entries
Collect completed diary card and left over medication
Recording of vital signs
Blood laboratory assessment
Recording of concomitant medication
Recording of any (serious) adverse events
Check on relapse to hormonal therapy
Drug accountability and check for compliance.
   Note: the Week 12 visit is not planned before Day 85.
   Premature discontinuation from treatment the Week 12 assessments are performed in case of premature discontinuation.

### Post-Treatment Period

Week 13 Visit upon request of the treated woman 1-30 days after the last dose of test medication in order to decide on the treatment result and the effect of non-treatment, followed by a decision to maintain non-treatment or to reinstate treatment with S-mirtazapine maleate.

Week 16 Visit: In case of maintained non-treatment 30-35 days after the last dose of test medication, the following assessments are performed:
Check on completion of daily diary entries
Collect completed diary card
Recording of vital signs
Recording of concomitant medication
Recording of any (serious) adverse events
Check on relapse to hormonal therapy.

1-7. (canceled)

8. A method to effectively wean a woman from hormonal therapy for treatment of vasomotor symptoms by reducing the dosage of the hormonal agent to zero, while a non-hormonal drug in an amount, which is therapeutically effective for treating vasomotor symptoms, is administered or initiated and continued for one or more periods of durations of 1-6 months whereby the number of periods is determined by assessment of the effect of a treatment free period of a duration of 1-7 days, which follows each period of continuous non-hormonal drug treatment.

9. The method according to claim 8, wherein the non-hormonal drug is S-mirtazapine or a salt thereof.

10. The method according to claim 8, wherein the woman is after her menopause.

11. The method according to claim 8, wherein the non-hormonal drug is S-mirtazapine or a salt thereof, which is administered in an amount of 3 or 4.5 mg S-mirtazapine per day.

12. The method according to claim 9, wherein the woman is after her menopause.

13. The method according to claim 9, wherein the S-mirtazapine or a salt thereof is administered in an amount of 3 or 4.5 mg S-mirtazapine per day.

14. The method according to claim 10, wherein the non-hormonal drug is S-mirtazapine or a salt thereof, which is administered in an amount of 3 or 4.5 mg S-mirtazapine per day.

15. The method according to claim 9, wherein the S-mirtazapine or a salt thereof is administered in an amount of from 1 to 10 mg per day.
